(19) United States
(12) Patent Application Publication
(10) Pub. No.: US 2020/0375894 A1
(43) Pub. Date: Dec. 3, 2020

(54) ANTI-BACTERIAL FOOD ADDITIVE AND ANTI-BACTERIAL FOOD INCLUDING THE SAME

(71) Applicant: Forever Co., Ltd., Kawaguchi-shi (JP)

(72) Inventor: Akira Hirai, Kawaguchi-shi (JP)

(73) Assignee: Forever Co., Ltd.

(21) Appl. No.: 16/548,821

(22) Filed: Aug. 22, 2019

(30) Foreign Application Priority Data
Jun. 3, 2019 (JP) 2019-104047

(51) Int. Cl. A61K 9/68; A61K 33/38; A61K 9/14

(52) U.S. Cl. CPC A61K 9/0058; A61K 9/14; A61K 33/38

(57) ABSTRACT

The present invention relates to an anti-bacterial food additive and anti-bacterial food including the same, and more particularly, to an anti-bacterial food additive which includes a sintered silver-containing surface layer disposed on a base for a food additive, wherein the sintered silver includes silver sintered in a nitrogen atmosphere or silver oxide sintered in an oxygen atmosphere. In addition, anti-bacterial food including the anti-bacterial food additive has an excellent anti-bacterial effect.

### CROSS-REFERENCE TO RELATED APPLICATION

This application claims priority to and the benefit of Japanese Patent Application No. 2019-104047, filed on Jun. 3, 2019, the disclosure of which is incorporated herein by reference in its entirety.

### TECHNICAL FIELD

The present invention relates to an anti-bacterial food additive and an anti-bacterial food including the same, and more particularly, to an anti-bacterial food additive which includes a sintered silver-containing surface layer disposed on a base for a food additive, in which the sintered silver includes silver sintered in a nitrogen atmosphere or silver oxide sintered in an oxygen atmosphere.

### TECHNICAL BACKGROUND

Xylitol-containing gum, which is an example of anti-bacterial food, has an anti-bacterial effect on oral diseases including dental caries. In fact, since early 1970s, xylitol has been proven to be effective in dental caries and other oral diseases through various in vivo or in vitro experiments and clinical and physical investigations. However, in the case of xylitol-containing gum, it is insufficient to experience the anti-bacterial effect.

In dental medicine, various oral diseases including dental caries, gingivitis and periodontitis may occur by various causes. The causes may be classified into systemic causes and local causes, and the local causes are known as critical causes. The critical cause of the local causes is bacteria that reside in the oral cavity, that is, pathogenic microorganisms.

Meanwhile, tens of thousands of microorganisms are widely inhabited in various types of ecosystems in nature, and these microorganisms are also responsible for various diseases and contaminations. For example, in the oral cavity of a human, it has been known so far that about 300 types of microorganisms reside on the tooth surface, between teeth and gum at a dental root and on the surface of the tongue, and the presence of these microorganisms may be a normal phenomenon when appropriate oral hygienic activities are performed.

However, when appropriate oral hygienic activities are not performed, among the above-mentioned microorganisms, by pathogenic microorganisms, various oral diseases such as dental caries, halitosis, gingivitis or periodontal disease may be caused, and when the diseases become serious, teeth may be lost. As an example, dental caries may occur through breakdown of the enamel of the hard tissues of teeth due to lactic acids made by decomposing carbohydrates such as sugar or starch in food debris attached to the teeth due to the fermentation by pathogenic microorganisms residing in the oral cavity, and large-scale proliferation of anaerobic pathogens may be the cause of various oral diseases.

Pathological microorganisms residing in the oral cavity form bacterial populations called dental plaque only a few hours after attaching to the dental surface and are proliferated. At first, while the dental plaque is formed when the pathological microorganisms are intensively attached on the tooth surface at the upper part of the gum which is seen with eyes, as the formation of the plaque is gradually progressing, dental plaque is also formed on the tooth surface at the lower part of the gum. When the dental plaque is formed on the tooth surface, the pathological microorganisms produce acids using sugar entering the oral cavity, and the acids may break down minerals which are the main components of a tooth and thus break teeth, resulting in dental caries.

Meanwhile, bacteria residing in dental plaque at the lower part of the gum, particularly, anaerobic, Gram-negative microorganism, secrete toxins and proteolytic enzymes to directly break down periodontal tissues or react with immune cells of the human body, thereby inducing the production of various immune materials, and these materials may lead to inflammation and breakdown of periodontal tissues.

There is the seriousness in that teeth and periodontal tissues can be treated once being destroyed, but they are not restored to their original tissue condition, and for this reason, fundamental blocking of the process of growing pathological microorganisms, which is the representative local cause of the destruction, in the oral cavity may be a method of preventing or rapidly treating an oral disease.

Examples of the microorganisms causing such oral diseases include Streptococcus mutans, Porphyromonas gingivalis, etc. The Streptococcus mutans is the causative bacterium of dental caries, which causes various oral diseases. Particularly, glucan and lactic acid produced during the growth of Streptococcus mutans destruct the enamel of the teeth and generate plaque, thereby causing dental caries.

Conventionally, sodium copper chlorophyllin as an anti-bacterial enzyme, a composite containing a fluorine or chlorine component such as sodium fluoride or benzethonium chloride, an aromatic carboxylic acid including benzoic acid, allantoin, tocopherol acetate, an antiplasmine or an antibiotic was used.

However, the composite containing a fluorine or chlorine component has a safety problem for a human body, and the antibiotic may have systemic side effects to the whole body, including diabetes, vomiting, etc., and lead to the occurrence of resistant bacteria in the oral cavity and induction of superinfection, and thus the antibiotic is difficult to be used for a long time, and can be used only as a therapeutic agent. Therefore, it is urgent to develop a natural material-derived composition for preventing or treating an oral disease which facilitates prevention or treatment, does not have a problem of resistance or stability, compared to an antibiotic, even when being used for a long time, and does not have a side effect, compared to a composite containing a fluorine or chlorine component. In addition, there is a limit to experience the immediate anti-bacterial effect of the conventional anti-bacterial food additives and enzymes.

[Prior Art] Korean Patent No. 10-1046621

### SUMMARY OF THE INVENTION

To improve a conventional problem in that it is difficult to experience the immediate anti-bacterial effect of anti-bacterial food, the present invention is directed to providing a food additive having an excellent anti-bacterial effect within a short period of time, for example, several minutes since including talc having a sintered silver-containing surface layer, and anti-bacterial food including the same.

However, technical problems to be solved in the present invention are not limited to the above-described problems, and other problems which are not described herein will be fully understood by those of ordinary skill in the art from the following descriptions.

One aspect of the present invention provides an anti-bacterial food additive which includes a base for a food additive, and a sintered silver-containing surface layer formed on the surface of the base, wherein the sintered silver is silver sintered in a nitrogen atmosphere or silver oxide sintered in an oxygen atmosphere.

The base for a food additive comprises talc particles. The sintered silver-containing surface layer disposed on the talc particles has a thickness of 0.1 to 100 μm. A weight ratio of the talc particles to the silver sintered in a nitrogen atmosphere or silver oxide sintered in an oxygen atmosphere is 100:0.1 to 50.

Another aspect of the present invention provides a method of preparing an anti-bacterial food additive, which includes: a first process of preparing a silver salt solution by adding and dissolving silver salt compound powder in water; a second process of coating a base for an anti-bacterial food additive with the silver salt solution; and a third process of forming a sintered silver-containing surface layer on the base for an anti-bacterial food additive by sintering the silver salt compound applied on the base for an anti-bacterial food additive at 440 to 500° C. in a nitrogen atmosphere or an oxygen atmosphere.

In the first process, 1 to 10 parts by weight of the silver salt compound powder is added to 100 parts by weight of the water. The silver salt compound is selected from silver carbonate, silver chlorate, silver chromate, silver vanadate, silver manganate, silver nitrate, silver nitrite, silver perchlorate, silver phosphate, silver acetate, and a mixture thereof.

Still another aspect of the present invention provides anti-bacterial food including an anti-bacterial food additive. The food is one selected from the group consisting of gum, candy, yeot (taffy), chocolate, yanggaeng (sweet jelly of red beans), and marshmallow.

The gum comprises a gum base and a gum coating layer, in which the gum base is one or more selected from the group consisting of natural gum, polyisobutylene, polyvinylacetate, ester gum, a microcrystalline wax, a plasticizer and a bulking agent. The gum coating layer comprises one or more additives selected from the group consisting of a moisturizing agent, an emulsifier, a flavor and an acidulant.

### DETAILED DESCRIPTION OF EXEMPLARY EMBODIMENTS

Hereinafter, embodiments of the present invention will be described in detail such that the present invention can be easily implemented by those of ordinary skill in the art to which the present invention belongs. However, the present invention may be embodied in many different forms, and is not limited to the embodiments described herein.

Anti-Bacterial Food Additive

An anti-bacterial food additive of the present invention includes a sintered silver-containing surface layer disposed on a base for a food additive, wherein the sintered silver is silver sintered in a nitrogen atmosphere or silver oxide sintered in an oxygen atmosphere.

Specifically, the present invention is prepared by coating a talc-containing base for a food additive with silver, wherein the silver may be silver sintered in a nitrogen atmosphere or silver oxide sintered in an oxygen atmosphere. In addition, the present invention may also be comprehended by a method of coating talc with silver.

The sintered silver-containing surface layer may be a layer disposed on a sintered silver-containing surface layer (hereinafter, refers to a “surface layer containing silver sintered in a layered form at nitrogen atmosphere or silver oxide sintered in a layered form at oxygen atmosphere”), which includes silver sintered in a layered nitrogen atmosphere or silver oxide sintered in a layered oxygen atmosphere.

The surface layer containing silver sintered in a nitrogen atmosphere or silver oxide sintered in an oxygen atmosphere may be a layer disposed on the surface of a base for a food additive (hereinafter, referred to as “surface layer containing silver sintered in a particulate form at nitrogen atmosphere or silver oxide sintered in a particulate form at oxygen atmosphere”), which may include silver sintered in a particulate nitrogen atmosphere or silver oxide sintered in a particulate oxygen atmosphere, which is exposed to the surface of a base, and include silver sintered in a particulate nitrogen atmosphere or silver oxide sintered in a particulate oxygen atmosphere, which is exposed to the surface of a base, and silver sintered in a particulate nitrogen atmosphere or silver oxide sintered in a particulate oxygen atmosphere, which is dispersed in a base.

The surface layer containing the silver sintered in a nitrogen atmosphere or silver oxide sintered in an oxygen atmosphere on talc particles may have a thickness of 0.1 to 100 μm. In this range, an excellent anti-bacterial effect may be exhibited.

The silver sintered in a nitrogen atmosphere or silver oxide sintered in an oxygen atmosphere may be contained at 1 to 50 wt % with respect to the total weight of the surface layer containing the silver sintered in a layered nitrogen atmosphere or silver oxide sintered in a layered oxygen atmosphere. In this range, an excellent initial anti-bacterial activity is exhibited. The surface layer containing the silver sintered in a layered nitrogen atmosphere or silver oxide sintered in a layered oxygen atmosphere may include silver, a silver compound such as silver oxide, and other impurities, and further include other anti-bacterial components, other than the silver compound.

Talc is talcum powder with the chemical formula 3Mg.4SiO.H₂O, which has a grease-like luster with colors ranging from white to buff. A weight ratio of talc particles to the silver sintered in a nitrogen atmosphere or silver oxide sintered in an oxygen atmosphere may be 100:0.1 to 50, and preferably, 100:1 to 40.

When the surface layer containing silver sintered in a nitrogen atmosphere or silver oxide sintered in an oxygen atmosphere, disposed on the talc particles, includes the silver sintered in a nitrogen atmosphere and the silver oxide sintered in an oxygen atmosphere within the above-mentioned range, an excellent early anti-bacterial activity is exhibited.

The talc particles with the surface layer containing the silver sintered in a nitrogen atmosphere or silver oxide sintered in an oxygen atmosphere may have a diameter of 1 to 20 μm. In addition, other than talc, silicon dioxide or magnesium stearate can be used as a base for a food additive.

As an anti-bacterial food additive, the talc particles with the surface layer containing the silver sintered in a nitrogen atmosphere or the silver oxide sintered in an oxygen atmosphere are preferably contained at 0.03 wt % or more and 5 wt % or less with respect to the total weight of the anti-bacterial food composition. Here, food includes one selected from the group consisting of gum, candy, yeot (taffy), chocolate, yanggaeng (sweet jelly of red beans), and marshmallow.

Method of Preparing Anti-Bacterial Food Additive

A method of preparing an anti-bacterial food additive of the present invention includes: a first process of preparing a silver salt solution by adding and dissolving silver salt compound powder in water; a second process of coating a base for an anti-bacterial food additive with the silver salt solution; and a third process of forming a sintered silver-containing surface layer on the base for an anti-bacterial food additive by sintering the silver salt compound applied on the base for an anti-bacterial food additive in a nitrogen atmosphere or in an oxygen atmosphere. Here, in the first process, 1 to 10 parts by weight of the silver salt compound powder may be added to 100 parts by weight of water.

The silver salt compound may be selected from silver carbonate, silver chlorate, silver chromate, silver vanadate, silver manganate, silver nitrate, silver nitrite, silver perchlorate, silver phosphate, silver acetate, and a mixture thereof.

In the third process, a sintering temperature may be 440 to 500° C. The sintering temperature may vary according to the type of silver salt compound and the base for an anti-bacterial food additive.

The anti-bacterial food additive prepared as described above kills many harmful bacteria present in a part of the human body, particularly, in the oral cavity by silver ions eluted by moisture, and makes the oral cavity clean hygienically.

Anti-Bacterial Food

An anti-bacterial food of the present invention may include an anti-bacterial food additive. Specifically, the anti-bacterial food may be one selected from the group consisting of gum, candy, yeot (taffy), chocolate, yanggaeng (sweet jelly of red beans), and marshmallow.

Particularly, the anti-bacterial food additive is preferably used for gum. While a person chews gum for a certain period of time, saliva comes, and thus an anti-bacterial activity is exhibited by the contact of the saliva to a sintered silver-containing coating layer of talc included in the gum. Therefore, by chewing the gum, bacteria in the mouth may be significantly reduced. Particularly, an excellent anti-bacterial effect on Streptococcus nutans or Porphyromonas gingivalis is exhibited. While the time for chewing gum may vary according to the type of gum base, and one or more minutes, preferably, three minutes or more, and more preferably, five minutes or more after chewing, the anti-bacterial effect may be exhibited.

The anti-bacterial gum includes a gum base and a gum coating layer. As the gum base, one or more selected from the group consisting of natural gum, polyisobutylene, polyvinylacetate, ester gum, a microcrystalline wax, a plasticizer and a bulking agent may be included, but the present invention is not limited thereto. For example, the gum base includes 5 to 15 wt % of natural gum, 5 to 20 wt % of polyisobutylene, 30 to 50 wt % of polyvinylacetate and 3 to 10 wt % of ester gum as a main component, and further includes 5 to 15 wt % of a microcrystalline wax, 5 to 15 wt % of a plasticizer, and 10 to 20 wt % of a bulking agent.

The natural gum may be used to provide suitable texture at the oral temperature, and include one or more selected from the group consisting of Chicle, Jelutong, Sorva and Sorvinha. The natural gum may be contained at 5 to 15 wt % with respect to the total weight of the gum base. When the natural gum is contained at less than 5 wt %, there is a limit to provide a sticky texture, and when the natural gum is contained more than 20 wt %, the gum is so much sticky and flexible to chew.

The polyisobutylene is used to provide stickiness and flexibility to gum tissue, and may be contained at 5 to 20 wt % with respect to the total weight of the gum base. When the polyisobutylene is contained at less than 5 wt %, there is a limit to provide a sticky texture, and when the polyisobutylene is contained at more than 20 wt %, the gum is so much sticky and flexible to chew.

The polyvinylacetate is used to provide flexibility and hardness to gum, and may be included at 30 to 50 wt % with respect to the total weight of the gum base. When the polyvinylacetate is included at less than 30 wt %, the gum texture becomes soft and vulnerable to external environments such as heat and moisture, resulting in leakage of the gum base, and when the polyvinylacetate is included at more than 50 wt %, the gum texture is not soft.

The ester gum is used to provide flexibility of the gum tissue and form a coating for bubble gum, and may be included at 3 to 10 wt % with respect to the total weight of the gum base. When the ester gum is contained at less than 3 wt %, the gum texture is not soft, and when the ester gum is contained at more than 10 wt %, the gum texture becomes soft.

The microcrystalline wax is used to reinforce a transition point of a natural resin and raise miscibility, and may be included at 5 to 15 wt % with respect to the total weight of the gum base. When the microcrystalline wax is contained at less than 5 wt %, there is a limit to maintain a soft texture, and when the microcrystalline wax is contained at more than 15 wt %, there is a limit in improving the miscibility of a flexible polymer and a resin.

The plasticizer serves as a softening agent imparting softness of the gum base, and may include 5 to 15 wt % with respect to the total weight of the gum base. When the plasticizer is contained at less than 5 wt %, the gum base becomes hard or syrup may be leaked due to loose joints in stamping of the gum base, and when the plasticizer is contained at more than 15 wt %, the outer part of the gum base may be vulnerable to an outdoor temperature and humidity, resulting in the leakage of syrup.

The bulking agent may be calcium carbonate serving to increase the entire volume of gum, and may be contained at 10 to 20 wt % with respect to the total weight of the gum base. When the bulking agent is contained at less than 10 wt %, the volume of gum may be reduced, and when the bulking agent is contained at more than 20 wt %, the flexibility of gum is degraded.

The gum base of the present invention may include a flexible polymer (a) and a resin (b), wherein the flexible polymer (a) includes natural gum and polyisobutylene, and the resin (b) includes polyvinylacetate and ester gum. The flexible polymer and the resin are preferably used by mixing them in a weight ratio of 1:2 to 1:4. When the mixing ratio is less than 1:2, the gum tissue becomes loose, and when the mixing ratio is more than 1:4, the gum is too much hard, but not flexible, there may be a possibility of syrup leakage.

In addition, the gum base mixed within the above range may have a viscosity of 1,000,000 to 5,000,000 cp (Brookfield Viscometer, 110° C., 0.5 rpm) at room temperature (25° C.). When the viscosity is more than 5,000,000 cp, the gum texture may become hard, and when the viscosity is less than 1,000,000 cp, the gum texture may become soft.

The gum base is preferably contained at 20 to 40 wt % with respect to the total weight of the anti-bacterial gum. When the gum base is less than 20 wt %, the gum tissue becomes loose, and thus the gum base may be leaked, and when the gum base is more than 40 wt %, there may be problems on a texture and a preparation process, and joints between the gum bases become loose and thus leakage may occur.

The coating for the anti-bacterial gum may further include one or more additives selected from the group consisting of a moisturizing agent, an emulsifier, a flavor and an acidulant.

The talc particles with the sintered silver-containing surface layer may include any one or both of the gum base and the gum coating layer of the anti-bacterial gum. The talc particles with the sintered silver-containing surface layer are preferably used at 0.03 wt % to 5 wt % with respect to the total weight of the anti-bacterial gum, and when the talc particles with the sintered silver-containing surface layer are used at less than 0.03 wt %, an anti-bacterial effect may be reduced, and when the talc particles with the sintered silver-containing surface layer are used at 5 wt %, the texture of the gum may be bad or there is a limit to be used as a gum composition. In addition, other than talc, silicon dioxide or magnesium stearate can be used as a base for a food additive.

In addition, as needed, sugar, starch syrup, fructose or invert sugar, or a mixture thereof, and distilled water may be included as additives for gum.

An anti-bacterial gum composition according to the present invention is prepared by methods such as the measurement, input, mixing, ejection, molding, stamping, cooling, aging and packaging of raw materials, and there is no limit to a method that can be applied in the art.

Hereinafter, the present invention will be described in further detail with reference to experimental examples of the present invention, and the range of the present invention is not limited to the following experimental examples.

### [Experimental Example] Evaluation of Anti-Bacterial Activity of Test Solution Including Anti-Bacterial Food Additives of the Present Invention

Next, an anti-bacterial experiment for a ball having a sintered silver-containing coating layer of the present invention was performed. The experiment was carried out in the Japan Food Research Laboratories (JFRL).

Porphyromonas gingivalis and Streptococcus nutans test bacteria were cultured in normal agar media (Eiken Chemical Co., Ltd.) at 35° C. (±1° C.) for 18 to 24 hours, and suspended in distilled water (a saline solution for Staphylococcus aureus) to have a cell density of 10⁷ to 10⁸ cells/mL, resulting in obtaining a test cell culture. Specimens are as follows.

(Specimen) Ball having sintered silver-containing coating layer

(Control) Distilled water contained in sterilized synthetic resin container

(Test solution) A test solution was prepared by inoculating a sample prepared by adding 200 mL of mineral water to the specimen subjected to dry heat sterilization (170° C., 1 hour) with 2 mL of the test cell culture. Six hours and 24 hours after storage at 20° C. (±1° C.), the test solution was immediately diluted 10 fold using an SCDLP medium (Nihon Pharmaceutical Co., Ltd.), and then a viable cell count in the test solution was measured using a medium for measuring a bacterial cell count.

The test result is shown in Tables 1 and 2 below. The anti-bacterial test showed that the specimen exhibited a sufficient anti-bacterial effect on Porphyromonas gingivalis and Streptococcus mutans. Table 1 shows the result of measuring a viable cell count over time using Porphyromonas gingivalis test bacteria in the test solution, and Table 2 shows the result of measuring a viable cell count over time using Streptococcus mutans test bacteria in the test solution.

TABLE 1
Result of measuring viable cell count in test solution

| | | Viable cell count (/ml) | | | | | |
|---|---|---|---|---|---|---|---|
| | Object for | After | After 5 | After 10 | After 15 | After 2-20 | After 25 |
| Test bacteria | measurement | initiation | minutes | minutes | minutes | minutes | minutes |
| Porphyromonas | Specimen* | — | 2.4 × 10⁵ | 30,000 | 10,000 | <10,000 | <10,000 |
| gingivalis | Control | 6.4 × 10⁵ | 3.0 × 10⁵ | 1.6 × 10⁵ | 1.1 × 10⁵ | 6.1 × 10⁵ | 1.8 × 10⁵ |

*(<10,000: indicating that the bacteria were not detected), Storage temperature: 20° C.

Referring to Table 1, it can be confirmed that the test solution of the present invention was tested to measure a viable cell count for Porphyromonas gingivalis, which was apparently different from the control from 5 to 10 minutes after the test. From 20 minutes after the test, it was confirmed that almost no viable cell count was detected.

TABLE 2
Result of measuring viable cell count in test solution

| | | Viable cell count (/ml) | | | | | |
|---|---|---|---|---|---|---|---|
| Test | Object for | After | After 5 | After 10 | After 15 | After 20 | After 25 |
| bacteria | measurement | initiation | minutes | minutes | minutes | minutes | minutes |
| Streptococcus | Specimen* | — | 4.7 × 10⁴ | 1.2 × 10⁴ | 2.4 × 10² | 30 | <10 |
| mutans | Control | 7.1 × 10⁵ | 6.3 × 10⁵ | 6.3 × 10⁵ | 6.0 × 10⁵ | 6.2 × 10⁵ | 6.2 × 10⁵ |

*(<10,000: indicating that the bacteria were not detected), Storage temperature: 20° C.

Referring to Table 2, it can be confirmed that the test solution of the present invention was tested to measure a viable cell count for Streptococcus mutans, which was apparently different from the control from 10 to 15 minutes after the test, and from 20 minutes after the test, it was confirmed that almost no viable cell count was detected.

Subsequently, to investigate how talc having a sintered silver-containing coating layer is effective on the oral cavity, a silver ion detection test was carried out. A silver raw material used herein was an aqueous solution of silver nitrate powder. In addition, the talc used herein was food additive No. 194 mineral talc. First, a solution was prepared by inputting 100 g of silver nitrate powder into 1 L water. Afterward, talc powder coated with a sintered silver-containing coating layer was obtained by preparing a mixture of talc powder and the food additive silver solution and sintering the mixture by increasing heat to average 450° C. Then, the gum was mixed with the talc powder coated with the sintered silver-containing coating layer at a weight ratio of 99.95:0.05.

The talc powder-containing gum prepared as described above was chewed in the mouth, and then a silver ion value eluted from the gum including the talc powder coated with the sintered silver-containing coating layer in the mouth was measured using a silver ion content meter. Five minutes after chewing in the mouth, the silver ion content was 0.01 ppm, and 10 minutes after chewing, the silver ion content was 0.02 ppm. Therefore, it can be confirmed that the anti-bacterial effect of silver ions on the oral harmful bacteria such as Porphyromonas gingivalis and Streptococcus nutans was confirmed by measuring the silver ion content. As a result, it was confirmed that anti-bacterial food containing the anti-bacterial food additive of the present invention exhibited an excellent anti-bacterial effect.

An anti-bacterial food additive of the present invention and anti-bacterial food including the same have an excellent anti-bacterial effect to food, particularly, gum, candy, yeot (taffy), chocolate, yanggaeng (sweet jelly of red beans), and marshmallow.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

What is claimed is:

1. An anti-bacterial food additive comprising a base for a food additive, and a sintered silver-containing surface layer formed on the surface of the base, wherein the sintered silver is silver sintered in a nitrogen atmosphere or silver oxide sintered in an oxygen atmosphere.

2. The anti-bacterial food additive according to claim 1, wherein the base for a food additive comprises talc particles.

3. The anti-bacterial food additive according to claim 2, wherein the sintered silver-containing surface layer disposed on the talc particles has a thickness of 0.1 to 100 μm.

4. The anti-bacterial food additive according to claim 2, wherein a weight ratio of the talc particles to the silver sintered in a nitrogen atmosphere or silver oxide sintered in an oxygen atmosphere is 100:0.1 to 50.

5. A method of preparing an anti-bacterial food additive, comprising:
a first process of preparing a silver salt solution by adding and dissolving silver salt compound powder in water;
a second process of coating a base for an anti-bacterial food additive with the silver salt solution; and
a third process of forming a sintered silver-containing surface layer on the base for an anti-bacterial food additive by sintering the silver salt compound applied on the base for an anti-bacterial food additive at 440 to 500° C. in a nitrogen atmosphere or in an oxygen atmosphere.

6. The method according to claim 5, wherein, in the first process, 1 to 10 parts by weight of the silver salt compound powder is added to 100 parts by weight of the water.

7. The method according to claim 5, wherein the silver salt compound is selected from silver carbonate, silver chlorate, silver chromate, silver vanadate, silver manganate, silver nitrate, silver nitrite, silver perchlorate, silver phosphate, silver acetate, and a mixture thereof.

8. Anti-bacterial food comprising an anti-bacterial food additive of claim 1.

9. The anti-bacterial food according to claim 8, wherein the food is one selected from the group consisting of gum, candy, yeot (taffy), chocolate, yanggaeng (sweet jelly of red beans), and marshmallow.

10. The anti-bacterial food according to claim 9, wherein the gum comprises a gum base and a gum coating layer, in which the gum base is one or more selected from the group consisting of natural gum, polyisobutylene, polyvinylacetate, ester gum, a microcrystalline wax, a plasticizer and a bulking agent.

11. The anti-bacterial food according to claim 10, wherein the gum coating layer comprises one or more additives selected from the group consisting of a moisturizing agent, an emulsifier, a flavor and an acidulant.
